(19) United States
(12) Patent Application Publication
(10) Pub. No.: US 2004/0077933 A1
(43) Pub. Date: Apr. 22, 2004

(54) Method and system for detecting radiation from wireless communication devices employing microorganisms

(76) Inventors: Eliora Ron, Tel Aviv (IL); Falk Fish, Tel Aviv (IL); Yossi Paitan, Reshon Lezion (IL); Iftach Jacoby, Kfar Hess (IL); Ran Rosen, Ein Vered (IL)

Correspondence Address: Gary M Nath, Nath & Associates, Sixth Floor, 1030 15th Street N W, Washington, DC, 20005, US

(21) Appl. No.: 10/465,916

(22) PCT Filed: Dec. 27, 2001
(86) PCT No.: PCT/IL01/01213

(51) Int. Cl. A61B 5/00

(52) U.S. Cl. 600/300

(57) ABSTRACT

A method and system for detecting or quantifying biologically effective radiation emitted from an activated wireless communication device such as a cellular phone or the antenna of a cellular phone. The method uses microorganismal cells producing a detectable response when exposed to biologically effective radiation. The device is placed a predetermined distance from microorganismal cells and then activated for a predetermined amount of time. At the end of the predetermined amount of time, it is determined whether the response was produced in the cells. Production of the response indicates that the device emitted biologically effective radiation.

### FIELD OF THE INVENTION

The invention is in the field of the detection of radiation.

### PRIOR ART

The following is a list of publications which is intended for better understanding of the Background of the Invention:

Blackman C. F., Benane S. G., Weil C. M., Ali J. S., “Effects of nonionizing electromagnetic radiation on single-cell biologic systems”. Ann. N. Y Acad. Sci. 274: 352-66, 1975.

Brusick D. R. Albertini, D. McRee, D. Peterson, G. Williams, P. Hanawalt, J. Preston, “Genotoxicity of radiofrequency radiation.” Environmental and Molecular Mutagenesis, 32(1):1-16, 1998.

Chagnaud, J L, Moreau, J M, Veyret, B, “No effect of short-term exposure to GSM-modulated low-power microwaves on benzo(a)pyrene-induced tumours in rat.” Int. J Radiat. Biol. 75(10):1251-1256, 1999.

Chipley J. R., “Effects of microwave irradiation on microorganisms”. Adv. Appl. Microbiol. 26:129-45, 1980.

Concar., D. (1999) “Get your head around this . . . ”, New Scientist, 10 Apr. 1999

Donner, M., P. Andrews, J. Clancy, D. Satterfield, M. Vasquez, R. R. Tice, J. McDougal, C. K. Chou, 0. J. Hook, A. W. Guy and D. McRee, “Genotoxicity Of 837 Mhz Radiofrequency Radiation (RFR) in A Battery Of In Vitro Bacterial And Mammalian Cell Assays (Poster 65)”. Environmental and Molecular Mutagenesis, 31(Suppl 29):40, Poster 65, 1998.

Dreyfuss M. S., Chipley J. R., “Comparison of effects of sublethal microwave radiation and conventional heating on the metabolic activity of Staphylococcus aureus.” Appl. Environ. Microbiol 39:13-6, 1980.

Drokina, T. V.; Popova, L. Yu, “Effect of millimeter electromagnetic waves on luminescence of bacteria.” Biofizika, 43(3): 522-525, 1998.

Kosted, P.; Rogers, S. J., “Methods to detect the effects of electromagnetic fields in biological systems.” Environmental and Molecular Mutagenesis, 27 (Suppl 27):37, 1996.

Kuster, N. and Schönborn, “Recommended minimal requirements and development guidelines for exposure setups of bio-experiments addressing the health risk concern of wireless communications”, Bioelectromagnetics, 21:508-514, 2000 Wiley-Liss, Inc.

McCann, J.; Dietrich, F.; Rafferty, C., “The genotoxic potential of electric and magnetic fields: an update.”, Mutat Res, 411(1): 45-86, 1998 (Quillardet P, Huisman, O, D'Ari, R., Hofnung, M., “SOS chromotest, a direct assay of induction of an SOS function in Escherichia coli K-12 to measure genotoxicity” Proc. Natl. Acad. Sci., USA, 79(19):5971-5 (1982)).

Miller J, Experiments in Molecular Genetics p. 48, Cold Spring Harbor Laboratory Cold Spring Harbor, N.Y., (1992).

Morandi, M. A.; Pak, C. M.; Caren, R. P.; Caren, L. D., “Lack of an EMP-induced genotoxic effect in the Ames assay.” Life Sci. 59(3): 263-71, 1996. Pascal Gos, Bernhard Eicher, Jurg Kohli, Wolf-Dietrich Heyer, “No mutagenic or recombinogenic effects of mobile phone fields at 900 mHz detected in the yeast Saccharomyces cerevisiae” Bioelectromagnetics”, 21:515-523, 2000.

Muscat, Joshua, M P H; Malkin, Mark G, MD, FRCPC; Thompson, Seth, Ph.D.; Shore, Roy E., Ph.D.; Stellman, Steven D., Ph.D.; McRee, Don, Ph.D.; Neugut, Alfred, I, Ph.D.; Wynder, Ernst L., MD: “Handheld Cellular Telephone Use and Risk of Brain Cancer”, JAMA, 284:3001-3007, 2000.

Phillips, L. A.; Blackwell, D. B.; Clancy, J. J; Donner, E. M.; Tice, R. R.; Hook, G. J., and McRee D. “Genotoxicity of radio frequency fields generated from analog, TOMA, CDMA, and PCS technology evaluated using a three test in vitro battery.” Abstract 159 of 1999 Environmental Mutagen Society Meeting, Washington, D.C., USA, Mar. 27-Apr. 1, 1999. Environmental And Molecular Mutagenesis, 33(SUPPL. 33):49, 1999.

Quillardet P, Huisman, O, D'Ari, R., Hofnung, M., “SOS chromotest, a direct assay of induction of an SOS function in Escherichia coli K-12 to measure genotoxicity” Proc. Natl. Acad. Sci., USA, 79(19):5971-5 (1982).

Repacholi M H; Basten A; Gebski V; Noonan D; Finnie J; Harris A W, “Lymphomas in E mu-Pim1 transgenic mice exposed to pulsed 900 MHz electromagnetic fields”. Radiat. Res., 147(5):631-40, 1997.

Time Europe Magazine, “Check the Label”, 25 Sep. 2000, page 32.

### BACKGROUND OF TIE INVENTION

With the increasing popularity of cellular phones there is growing concern with a putative health hazard posed by their radiation. Cellular communications employ UHF and microwave radio frequencies, similar to those employed in television sets and microwave ovens. In low doses, radio frequency electromagnetic radiation is considered safe and non-hazardous, being far removed from higher frequency, ionizing electromagnetic radiation, such as UV, X-rays and gamma-rays, that are known to damage biological systems.

Although the radiation emitted from cellular telephones and microwave ovens is believed to be “non-ionizing” and thus safe , some researchers have documented effects of non-ionizing electromagnetic radiation on bacteria. Chipley (1980), in a review of the scientific literature on the effect of microwave radiation on bacteria, pointed out inconsistencies among various reports: while some noted a lethal effect of such radiation on bacteria, others found either no effect or stimulation of growth (Blackman et al., 1975). When isolating the thermal effects from the non-thermal effects of microwave radiation, Dreyfuss and Chipley (1980) demonstrated an increase in the activity of malate and α-ketoglutarate dehydrogenases, cytochrome oxidase and cytoplasmic ATPase, while the activity of other enzymes either decreased or was unaffected.

In their concern with the potential hazard of radiation, cellular telephone manufacturers have consulted with government authorities in order to devise a global standard for measuring so-called “Specific Absorption Rates (SAR)”, which are a measure of radiation absorbed by human tissue (Time Europe, 2000). However, reliable means are needed for evaluating the biological effect of radiation emitted by a telephone unit. One of the reasons for a lack of unequivocable conclusions on the biological effect of such radiation is that there is no established standard for testing conditions which is essential for the design of interpretable and repeatable tests (Kuster et al., 2000).

Thus, after a large amount of research on the harmful effects of wireless communication devices the evidence is contradictory (Concar 1999) and a definitive conclusion has not yet been arrived at. For example, researchers at the Royal Adelaide Hospital in Australia have shown an increased incidence of lymphoma in mice exposed for 18 months to radiation simulating radiation emitted by cellular telephones (Repacholi et al, 1997). Others have not been able to repeat this observation (e.g. Chagnaud et al, 1999, Concar 1999). Other research showed that the use of handheld cellular telephones is not associated with a risk of brain cancers (Muscat et al., 2000).

Various attempts have been made to employ bacteria to evaluate the biological effects in general, and the genotoxic effects in particular, of radio-frequency radiation (Brusick et al., 1998; Donner et al., 1998; Phillips et al., 1999). However, these reports could not demonstrate a clear definite deleterious effect of electo-magnetic fields on bacterial DNA (Kosted and Rogers, 1996; Morandi et al. 1996; McCann et al., 1998; Kuster et al., 2000) and further failed to demonstrate any genotoxicity of cellular telephone frequency radiation (Brusick et al., 1998; Phillips et al., 1999). Conversely, Drokina and Popova (1998) noted an increase in light emission from luminescent marine bacteria exposed to millimeter-wave radiation. This effect however was dependent on the state of the bacterial culture, making it impossible to draw conclusions regarding any deleterious or beneficiary effects of cellular telephone radiation. Kosted and Rogers (1996) detected a decrease in β-galactosidase synthesis in bacteria carrying recA:lacZ fusion, that were subjected to radiation. Any damage to cellular DNA would have caused an increase of β-galactosidase synthesis.

### GLOSSARY

Throughout the specification, the following terms are used which, in accordance with the invention, should be understood to mean the following:

“Detecting”—noting changes in one or more characteristics of a microorganism expressed as a detectable signal generated by the microorganism in response to its exposure to a device producing radiation as compared to the same one or more characteristics in control microorganisms. The generated signal in the microorganisms may be any detectable signal such as a light, color or electric signal. These may be detected by any of the methods known in the art such as for example by colorimetric assays, enzymatic assays (such as enzyme linked immunoassays ELISA), radioactive assays, light emission, fluorescence, electrochemistry etc).

“Quantifying”—measuring the level of one or more signals generated in microorganisms contacted with a tested radiation device and comparing the level of said one or more signals to the level of the same signal in microorganisms either not contacted with the tested device or contacted with a substance known to cause a reaction in said cells. The quantification may be carried out using any of the methods known in the art such as those mentioned above.

By one embodiment, quantification of the radiation effect involves analyzing its effect on cells placed at different distances from a radiation device and measuring the effect of the emitted radiation on the cells at each distance. In accordance with this embodiment, the microorganisms may be arranged in multiplex arrays and exposed to the radiation. In a multiplex array are cells placed at various distances from the radiation source. In addition the array may include several kinds of microorganismal cell types in a single test. In addition, the cells may be arranged in multi-dimensional arrays which enable testing of the spatial effect of the radiation on the cells, as well as additional factors such as return radiation, etc.

“Biologically effective radiation”—any radiation emitted from a tested device which may have an effect on biological systems.

“Wireless communication device”—any device which is operated wireless, being mainly cellular telephones as well as stationary antennas, transmitters, etc.

“Activated”—refers to a mode of the device in which it emits radiation. When the device is a cellular telephone, its activation may be by placing a call to the telephone or by calling from it.

“Microorganisms” (“microorganismal cells”)—includes bacterial cells, unicellular eukaryotic cells such as yeast cells, algae, protozoa, fungi, etc. The reaction of such cells to radiation is expressed in the generation of one or more signals (color, light, electric, fluorescent etc.) that are detectable or quantifiable by methods known in the art. The reaction may be any physiological reaction such as changes in the cell membrane, genetic changes and changes in the expression level of a gene. By a preferred embodiment, the microorganisms are bacterial cells constructed to be responsive to radiation which elicits detectable changes in the cells. The microorganismal cells will at times be referred to herein as “indicator cells”, being indicative of radiation emitted from a tested device.

### SUMMARY OF THE INVENTION

In accordance with the invention it has surprisingly been found that it is possible to detect and measure biologically effective radiation emitted from wireless communication devices by employing microorganisms. The invention is based on findings showing that exposure of bacterial cells sensitive to biologically effective radiation to radiation emitted by an activated cellular phone may result in genetic changes in the bacterial cells which may be detected and measured as an indication of the effect of the radiation. In accordance with the invention, the effect of the radiation is detected by exposing the microorganismal cells to a telephone itself rather than exposing the cells to simulated radiation fields. Thus, the radiation which is detected and measured is the actual radiation to which a cellular phone user would be exposed to. Moreover, the method of the invention reflects the components of the tested device and their effect on the radiation emitted from the device. In addition, the effect of the radiation emitted from the tested device may be determined within a very short period of time which ranges from less than an hour to several hours thus making the method suitable for screening a large number of devices within a short period of time.

Thus, the invention provides a method for detecting or quantifying biologically, emitted from activated wireless communication device comprising:

(a) placing the device a predetermined distance from microorganismal cells for a predetermined amount of time, the microorganismal cells producing a detectable response when exposed to biologically effective radiation; and

(b) determining whether the response was produced by the cells, a response being produced being indicative of biologically effective radiation being emitted by the device.

By a preferred embodiment, the wireless device is a cellular telephone, or the antenna of a cellular phone. In accordance with this embodiment, the cells are placed in wells of a micro-well plate, and the telephone is placed at the predetermined distance to the microorganismal cells so that the whole antenna is within the borders of the plate and the phone is then activated for the predetermined amount of time. The predetermined distance between the device and the cells is preferably less than one meter. In a more preferred embodiment, the distance is less than to centimeters. In a most preferred embodiment, the distance is less than three centimeters. The predetermined amount of time is preferably less than three hours. In a more preferred embodiment, the predetermined amount of time is less than one hour. In a most preferred embodiment, the predetermined amount of time is 30 minutes.

In accordance with the invention, the microorganismal indicating cells may either be cells which are capable of reacting to irradiation or, alternatively cells which are constructed to be sensitive to such radiation. The cells may be constructed to be sensitive to radiation by introducing genetic material encoding for a product which is expressed at a higher or lower level when the cells are exposed to the radiation. Such genetic material may be introduced into the cells by methods known in the art such as for example, by transfection, as described below.

By a preferred embodiment, the microoganismal cells are bacterial cells. As mentioned above, the bacterial cells may be reactive to irradiation in their natural form or, alternatively, constructed to become sensitive to such radiation. One such example of constructed bacteria are E.coli cells comprising the pC-RB-C2 plasmid (in which the LacZ gene is fused to the promoter of the dnaK gene which encodes for the heat shock protein HSP70). These cells respond in a calorimetric reaction to stress (as shown in the examples below).

The invention also provides a system for detecting or quantifying biologically effective radiation emitted from a wireless communication device comprising:

(a) microorganismal cells producing a detectable response when exposed to biologically effective radiation; and

(b) a detector for detecting the response in the cells.

The system may also optionally comprise an apparatus for exposing the cells to radiation emitted from a wireless communication device.

The invention also provides a kit for use in the detection or quantification of biologically effective radiation emitted from a wireless communication device comprising:

(a) microorganismal cells producing a detectable response when exposed to biologically effective radiation; and

(b) a detector for detecting the response in the cells.

(c) a standard substance being a substance known to cause the result in the cells, and

(c) instructions for use.

The substance which is known to cause a reaction in the indicating cells serves as a positive control to verify that the cells included in the kit are indeed functional and capable of reacting. An example of such a substance is a carcinogenic compound known to cause detectable mutations in the indicating cells. The kit may also optionally comprise an apparatus for exposing the cells to radiation emitted from a wireless communication device.

### DETAILED DESCRIPTION OF THE INVENTION

The invention will now be demonstrated by the following non-limiting examples:

### EXAMPLE 1

### Solutions, Media and Bacterial Strains

### LB Medium

| | | |
|---|---|---|
| Bacto ® tryptone | 10 | g |
| Bacto ® yeast extract | 5 | g |
| NaCl | 10 | g |
| Water to | 1000 | mL |

The medium was autoclaved at 120° C. for 30 mins.

### Z Buffer

| | | |
|---|---|---|
| Na₂HPO₄.7H₂O | 16.1 | g |
| NaH₂PO₄.H₂O | 5.5 | g |
| KCl | 0.75 | g |
| MgSO₄.7H₂O | 0.246 | g |
| Beta mercaptoethanol | 2.7 | ml |
| Water to | 1000 | mL |

O-Nitro Phenyl ββ-D-Galactoside henceforth ONPG) was dissolved to 1 mg/mL in Z-buffer before use.

### Bacterial Strains

E. coli PQ37 is a recombinant strain in which the LacZ gene is fused to an SOS promoter (recA), (Quillardet et al., 1982). As a result, the cells of this strain respond to damage in their DNA by de novo synthesis of β-galactosidase, which can be detected and measured by colorimetry, fluorescence or electrochemistry. PQ37 bacteria were propagated in LB medium.

E.coli strain MC4100 carries a pC-RB-C2 plasmid, in which the lacZ gene is fused to the promoter of the dnaK gene (which encodes for the heat-shock protein—HSP70). As such, cells of this strain respond in a colorimetric or electrochemical reaction to stress. MC-4100 bacteria were propagated in LB medium.

The pC-RB-C2 plasmid was generated as follows:

1. The dnaK promoter was obtained by genomic PCR from E. coli strain MC4100.

2. Using the appropriate primers, EcoRI (5′ primer) and BamHI (3′ primer) restriction sites were introduced into the transcriptional fusion vector pTL61T, carrying a promoterless lacZ gene.

3. The dnaKₚ::lacZ fusion was introduced into the low copy number pCL1920 plasmid to obtain the pC-RB-C2 plasmid. The low copy provides high signal to noise ratio to enable sensitive detection of stress signals.

### EXAMPLE 2

### Detection of Radiation Emitted by a Cellular Telephone Employing Bacteria

An E. coli PQ37 or MC-4100 bacterial culture, grown overnight in LB medium, was diluted into 25 mL of fresh LB medium to a turbidity of 20 Klett units (as determined in a Klett photometer, (Klett Summerson Manufacturing Co, NY, USA ). Incubation of that culture continued at 37° C. until it reached a turbidity of 80 Klett units. At this point, 0.75 mL aliquots of the culture were added to each well of two 24-well cluster tissue culture disposable plastic plates (Cel-Cult, Sterilin LTD, Hounslow, UK).

A cellular phone, either Ericsson T28S or Nokia 5110 (both GSM), was placed on one of the plates, so that the entire antenna was within the borders of the plate. The distance between the antenna and the cells was from 1 to 5 centimeters. A phone call was then placed to that telephone for 30 minutes, while the plate was incubating at room temperature.

The other plate served as non-treated control and was incubated in the same room at a distance of 7 meters from the tested cellular telephone.

The activity of the SOS or HSP promoter, following exposure to the cellular radiation, was determined by monitoring the activity of β-galactosidase. 600 μL samples were taken from each of the wells of the 24-well plate and mixed for 10 seconds with 120 μL chloroform. 50 μL of the chloroform-treated culture were transferred to wells of a 96-well, flat bottom microtitration plate. 155 μL of Z buffer with ONPG were then added to each well and the plate was incubated at 37° C. After 30 min, the reaction was stopped by addition of 85 μL 1M Na₂CO₃. The Optical Density (henceforth OD) of the wells at 420 nm and 550 nm was measured, employing a SpectraMax 190 microplate reader Molecular Devices, Sunnyvale, Calif., USA).

The turbidity of the bacterial culture in each well of the 24-well plates, was determined by diluting a 100 μL sample of the culture into 300 μL of LB medium and determining the OD at 600 nm. The Enzyme Units of each culture were calculated by the following equation (Miller, 1992, p.48).

$$\frac{1000 \times ( OD 420 - 1.75 \times OD550 )}{T \times V \times OD600}$$

wherein:

OD 420 is a measure of β-galactosidase activity and light scattering by cell debris

OD 550 is scattering by cell debris

OD 600 is turbidity of the untreated culture

Tx=30 minutes.

Vx=0.290 ml

Results of a 30-minute exposure to a Nokia telephone unit are summarized in Table 1.

TABLE 1
Effect of 30 minute exposure to cellular phone radiation on
the SOS and Heat Shock systems of E. coli cultures

| | | | Exposure Effect | |
|---|---|---|---|---|
| | | | (% increase over control) | |
| | Enzyme activity in | | Stress | |
| | MC-4100 | | (In CV-4100 | DNA |
| Exposure | cells | PQ37 cells | Cells) | (In PQ37 cells) |
| None | 1913 ± 245 | 87 ± 1 | 0 | 0 |
| (Control) | | | | |
| 30 minutes | 4581 ± 2032 | 132 ± 22 | 139 | 51 |

It is clear from the data in Table 1 that the radiation had an effect on the genetic material and on the expression of the heat shock protein and/or gene, thus showing that the bacterial cells were stressed by the radiation.

### EXAMPLE 3

### Quantification of the Effect of Radiation Emitted by a Cellular Telephone

In order to demonstrate that the invention can quantify the amount of radiation, the enzyme activity in the individual culture wells was calculated with regard to the distance of the wells from the antenna of the cellular telephone. Thus, while 6 culture wells were located directly under the antenna, 6 adjacent wells were located 2 cm to the right of the antenna. The distance between the antenna and the surface of the culture medium was about 2.5 cm for the wells located just under the antenna, and about 3.2 cm for the neighboring wells.

Table 2 presents the effect of the distance from the antenna on the average response of PQ37 bacteria to the radiation.

TABLE 2
The effect of distance from antenna on PQ37 response

| | | % increase | |
|---|---|---|---|
| Distance | Enzyme Activity | above control | Significance |
| Control | 87 ± 1 | 0 | P < 0.001 |
| 2.5 cm | 153 ± 7 | 75 | |
| 3.2 cm | 119 ± 10 | 36 | |

It is clear from the data in Table 2, that the response of the bacteria to the radiation increases as the distance between the antenna and the bacteria increases. Hence, this invention can quantify radiation level, in addition to being able to detect it.

1. A method for detecting or quantifying biologically effective radiation emitted from an activated wireless communication device comprising:
(a) placing the device a predetermined distance from microorganismal cells for a predetermined amount of time, the microorganismal cells producing a detectable response when exposed to biologically effective radiation; and
(b) determining whether the response was produced by the cells, a response being produced being indicative of biologically effective radiation being emitted by the device.

2. The method according to claim 1, wherein the predetermined amount of time is less than three hours.

3. The method according to claim 2, wherein the predetermined amount of time is less than 1 hour.

4. The method according to claim 3, wherein the predetermined amount of time is 30 minutes.

5. The method according to any one of the previous claims wherein the predetermined distance is less than 1 meter.

6. The method according to claim 5, wherein the predetermined distance is less than 10 centimeters.

7. The method according to claim 6, wherein the predetermined distance is less than 3 centimeters.

8. The method according to any one of the previous claims wherein the device is a cellular phone.

9. The method according to any one of the previous claims wherein the micro-organismal cells are bacterial cells.

10. The method according to claim 9 wherein the bacterial cells are E. coli PQ 37 cells, and the response is de novo synthesis of β-galactosidase.

11. The method according to claim 9 wherein the bacterial cells are E. coli strain MC4100 cells and the response is de novo synthesis of β-galactosidase.

12. The method according to any one of the above claims wherein the microorganismal cells are placed at various distances from the device.

13. A system for detecting or quantifying biologically effective radiation emitted from a wireless communication device comprising:
(a) microorganismal cells producing a detectable response when exposed to biologically effective radiation; and
(b) a detector for detecting the response in the cells.

14. The system according to claim 13 wherein the cells are bacterial cells.

15. The system according to claim 14 wherein the bacterial cells are E. coli strain PQ37 cells and the response is de novo synthesis of β-galactosidase.

16. The system according to claim 14 wherein the bacterial cells are E. coli MC4100 cells and the response is de novo synthesis of β-galactosidase.

17. A kit for use in the detection or quantification of biologically effective radiation emitted from a wireless communication device comprising:
(a) microorganismal cells producing a detectable response when exposed to biologically effective radiation;
(b) a detector for detecting the response in the cells;
(c) a standard substance being a substance known to cause the result in the cells; and
(d) instructions for use.
